(19) United States
(12) Patent Application Publication
(10) Pub. No.: US 2026/0354452 A1
(43) Pub. Date: Nov. 12, 2026

(54) A DISPOSABLE AEROSOL DELIVERY DEVICE

(71) Applicant: Nicoventures Trading Limited, London (GB)

(72) Inventors: Mark HARRIMAN, Bristol (GB); Richard HAINES, London (GB); Matthew HODGSON, Ramsbury (GB); Ping Chou CHEN, London (GB); Catalin Mihai BALAN, London (GB)

(21) Appl. No.: 19/514,981

(22) PCT Filed: Aug. 30, 2024
(86) PCT No.: PCT/GB2024/052272

§ 371 Date: Feb. 27, 2026

(30) Foreign Application Priority Data
Aug. 31, 2023 (GB) 2313237.6

(51) Int. Cl. A24F 40/10; A24F 40/42; A24F 40/485

(52) U.S. Cl. CPC A24F 40/10; A24F 40/42; A24F 40/485

(57) ABSTRACT

A disposable aerosol delivery device is disclosed. The device comprises a housing having a first portion defining a battery compartment within the housing to power the device. Additionally, the housing comprises a second portion having a liquid reservoir. The first and second portions of the housing are connected to each other and are configured to enable their irreversible separation.

### TECHNICAL FIELD

The present disclosure relates to a disposable aerosol delivery device.

### BACKGROUND

Certain tobacco industry products produce an aerosol which is inhaled by a user during use. For example, tobacco heating devices heat an aerosol generating substrate such as tobacco to form an aerosol by heating, but not burning, the substrate. Such tobacco industry products commonly include mouthpieces through which the aerosol passes to reach the user's mouth.

### SUMMARY

According to an aspect of the present disclosure, there is provided a disposable aerosol delivery device comprising a housing having a first portion defining a battery compartment within the housing to power the device, and a second portion comprising a liquid reservoir, wherein the first and second portions of the housing are connected to each other, and wherein the first and second portions are configured to enable their irreversible separation.

The first portion may comprise control circuitry to operate the device.

The second portion may comprise a heater and a liquid flow path for the flow of liquid from the reservoir to the heater.

The second portion may comprise a vapour flow path for the flow of vapourised liquid from the heater out of the device.

The disposable aerosol delivery device may comprise a battery in the battery compartment and the first and second portions may be configured such that the battery is removable from said battery compartment when said first and second portions are irreversibly separated.

The housing may comprise a breakable region, the housing may be separable into said first and second portions when said breakable region is broken.

The breakable region may be configured to break in response to the application of a snapping force on either side of the breakable region.

The breakable region may be configured to break in response to the application of a relative rotational force on either side of the breakable region.

The breakable region may comprise a line of weakness to facilitate breaking of the breakable region.

The breakable region may comprise a tear-off strip.

The disposable aerosol delivery device may comprise an outer wrap surrounding the housing, said first and second portions may be irreversibly separable by unwrapping said outer wrap.

The disposable aerosol delivery device may comprise terminals in the housing in electrical contact with corresponding terminals on the battery received in the battery compartment.

The housing may be configured such that the electrical connection is irreversibly disconnected when the first and second portions are separated.

The electrical connection may be irreversibly disconnected when the breakable region is broken.

The battery may be non-rechargeable.

### BRIEF DESCRIPTION OF THE DRAWINGS

Embodiments of the invention will now be described, by way of example only, with reference to the accompanying drawings, in which:

FIG. 1 is a schematic view of a disposable aerosol delivery device according to an embodiment of the invention.

### DETAILED DESCRIPTION

As used herein, the term “delivery system” is intended to encompass systems that deliver at least one substance to a user, and includes:
non-combustible aerosol provision systems that release compounds from an aerosol-generating material without combusting the aerosol-generating material, such as electronic cigarettes, tobacco heating products, and hybrid systems to generate aerosol using a combination of aerosol-generating materials.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine.

In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thp thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

In some embodiments, the substance to be delivered comprises a flavour.

As used herein, the terms “flavour” and “flavourant” refer to materials which, where local regulations permit, may be used to create a desired taste, aroma or other somatosensorial sensation in a product for adult consumers. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavour comprises menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour comprises flavour components extracted from cannabis.

In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucalyptol, WS-3.

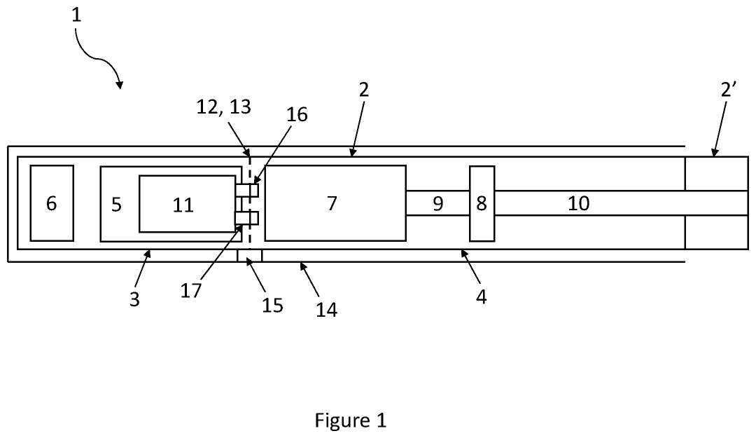

FIG. 1 illustrates a disposable aerosol delivery device according to an embodiment of the present invention. The device 1 comprises a housing 2, which includes a first portion 3 and a second portion 4, and a mouthpiece 2′. The first portion 3 of the housing 2 defines a battery compartment 5 to power the device 1 and comprises control circuity 6 to operate the device 1. The second portion 4 of the housing 2 comprises a liquid reservoir 7, a heater 8, and a liquid flow path 9 to allow the flow of liquid from the liquid reservoir 7 to the heater 8. Additionally, the second portion 4 of the housing 2 comprises a vapour flow path 10 for the flow of vapourised liquid from the heater 8 out of the device 1 through the mouthpiece 2′. The first and second portions 3, 4 of the housing 2 are configured to enable their irreversible separation after the device 1 is no longer usable.

As shown in FIG. 1, the device 1 may comprise a battery 11 in the battery compartment 5. When the first and second portions 3, 4 of the housing 2 are irreversibly separated, the battery 11 is removable from the battery compartment 5. In some embodiments, the battery is a non-rechargeable battery. This arrangement allows the battery to be removed easily from the device and recycled separately to the device.

In some embodiments, the housing 2 comprises a breakable region 12, which when broken, separates the housing 2 into the first and second portions 3, 4 described above and enables the user to access the battery 11 for disposal. The breakable region 12 may be configured to break in response to the application of a snapping force on either side of the breakable region 12. In other words, the user is able to break the housing 2 into the first and second portions 3, 4 by bending the two portions of the housing relative to each other about the breakable region 12. Alternatively, the breakable region 12 may be configured to break in response to a relative rotational force on either side of the breakable region 12. In other words, the user is able to separate the two portions of the housing by twisting the two portions of the housing relative to each other. Providing the housing with a breakable region which the user can manipulate in order to gain access to the battery within the battery compartment allows the user to remove the battery from the device without the need for any tools.

The breakable region 12 may comprise a line of weakness 13 in the material of the housing 2 to facilitate breaking of the breakable region 12. In some embodiments, the material of the housing 2 is continuous across the device 1 and the line of weakness 13 divides the housing 2 into the first and second portions 3, 4. Alternatively, the first and second portions 3, 4 of the housing 2 may be provided as two separate portions which are joined together within the breakable region 12 by a joining mechanism. The joining mechanism may be any suitable mechanism that is configured to break when the first and second portions 3, 4 of the housing 2 are separated. For example, the joining mechanism may comprise hooks, clips or a snap fit type mechanism which are irreversibly broken upon separation of the first and second portions 3, 4 of the housing 2. Additionally, in some embodiments, the breakable region 12 may comprise a tear off strip to enable tearing of the breakable region 12. The tear tape may comprise a pull tab to facilitate removal of the tear tape. In use, a user is able to grasp and pull the pull tab to remove the tear tape and thus break the breakable region.

In some embodiments, the device 1 comprises an outer wrap 14 surrounding the housing 2. The outer wrap 14 may be formed from a recyclable material, such as paper, cardboard or any other recyclable material in order to improve the recyclability of the device. In particular, the outer wrap 14 may be formed from a paper sheet. Additionally, it may be the case that the first and second portions 3, 4 of the housing 2 are irreversibly separable by unwrapping the outer wrap 14 from the device 1. The outer wrap 14 may comprise a connecting portion 15 which connects the outer wrap 14 to the housing 2. More specifically, the connecting portion 15 may connect the outer wrap 14 to the breakable region 12 such that removal of the outer wrap 14 breaks the breakable region 12. In this arrangement, once the device 1 is ready for disposal, the outer wrap 14 can be removed, breaking the breakable region 12, and the first and second portions 3, 4 of the housing 2 will separate, which in turn, enables the user to access and ultimately dispose of the battery 11 separate to the rest of the device 1.

In some embodiments, the housing 2 of the device 1 may comprise terminals 16 configured to be in electrical contact with corresponding terminals 17 on the battery 11 when the battery 11 is received within the battery compartment 5. When the first and second portions 3, 4 of the housing 2 are separated, the electrical connection between the terminals 16 in the housing 2 and the terminals 17 of the battery 11 may be irreversibly disconnected. In particular, the electrical connection may be irreversibly disconnected when the breakable region 12 of the housing 2 is broken. This arrangement ensures that the battery/device is not unintentionally activated after separation of the first and second portions of the device thereby improving safety during disposal.

The various embodiments described herein are presented only to assist in understanding and teaching the claimed features. These embodiments are provided as a representative sample of embodiments only, and are not exhaustive and/or exclusive.

It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

1. A disposable aerosol delivery device comprising a housing having a first portion defining a battery compartment within the housing to power the device, and a second portion comprising a liquid reservoir, wherein the first and second portions of the housing are connected to each other, and wherein the first and second portions are configured to enable their irreversible separation.

2. An aerosol delivery device according to claim 1, wherein the first portion comprises control circuitry to operate the device.

3. An aerosol delivery device according to claim 1, wherein the second portion comprises a heater and a liquid flow path for the flow of liquid from the reservoir to the heater.

4. An aerosol delivery device according to claim 2, wherein the second portion comprises a vapour flow path for the flow of vapourised liquid from the heater out of the device.

5. An aerosol delivery device according to claim 1, comprising a battery in the battery compartment and wherein the first and second portions are configured such that the battery is removable from said battery compartment when said first and second portions are irreversibly separated.

6. An aerosol delivery device according to claim 1, wherein the housing comprises a breakable region, the housing being separable into said first and second portions when said breakable region is broken.

7. An aerosol delivery device according to claim 6, wherein the breakable region is configured to break in response to the application of a snapping force on either side of the breakable region.

8. An aerosol delivery device according to claim 6, wherein the breakable region is configured to break in response to the application of a relative rotational force on either side of the breakable region.

9. An aerosol delivery device according to claim 6, wherein the breakable region comprises a line of weakness to facilitate breaking of the breakable region.

10. An aerosol delivery device according to claim 6, wherein the breakable region comprises a tear-off strip.

11. An aerosol delivery device according to claim 1, comprises an outer wrap surrounding the housing, said first and second portions being irreversibly separable by unwrapping said outer wrap.

12. An aerosol delivery device according to claim 1, comprising terminals in the housing in electrical contact with corresponding terminals on the battery received in the battery compartment.

13. An aerosol delivery device according to claim 12, wherein the housing is configured such that the electrical connection is irreversibly disconnected when the first and second portions are separated.

14. An aerosol delivery device according to claim 6, wherein the electrical connection is irreversibly disconnected when the breakable region is broken.

15. An aerosol delivery device according to claim 1, wherein the battery is non-rechargeable.
